(19) United States
(12) Patent Application Publication
(10) Pub. No.: US 2008/0033462 A1
(43) Pub. Date: Feb. 7, 2008

(54) Incision system for ophthalmology

(75) Inventors: Silvio Di Nardo, St. Gallen (CH); Bruno Nyffenegger, Balgach (CH)

Correspondence Address: THE WEBB LAW FIRM, P.C., 700 KOPPERS BUILDING, 436 SEVENTH AVENUE, PITTSBURGH, PA, 15219, US

(73) Assignee: OERTLI INSTRUMENTE AG, Berneck (CH)

(21) Appl. No.: 11/890,328

(22) Filed: Aug. 6, 2007

(30) Foreign Application Priority Data
Aug. 7, 2006 (EP) 06 118 554.2

(51) Int. Cl. A61F 9/007

(52) U.S. Cl. 606/166

(57) ABSTRACT

The present invention provides a pilot tube attachment that can be connected by coupling to a pilot tube inserted into an eye. The pilot tube attachment has a centre axis. The pilot tube attachment comprises an instrument guide and a clamp unit. The clamp unit comprises at least two catch elements which are arranged symmetrically with respect to the centre axis and which can be actuated by respectively associated pressure tabs via a transition leading into the instrument guide.

### TECHNICAL FIELD OF THE INVENTION

The present invention relates to an incision device for opthamology according to the features of the preamble of claim 1.

### PRIOR ART

Such devices are known from the prior art and are used by specialists in a large number of operations on the eye.

The Applicant markets such a device under the product name “Pars Plana Microincision System PMS”. The device comprises a large number of surgical instruments. Such a device is typically used for surgical interventions in an eye. A first trocar, also referred to as a pilot tube, is inserted into the eye in such a way that the tip of the trocar protrudes into the vitreous body (corpus vitreum) of the eye, while the opposite end is available as a line attachment. This opposite end can be connected to a line through which the interior of the eye can be supplied with an infusion via the first trocar, for which reason the trocar can also be referred to as an infusion trocar. Moreover, two further trocars are also normally used. Suitable instruments or illuminating devices can then be delivered to the eye through these further trocars. These further trocars are also referred to as instrument trocars or illuminating trocars.

This device is eminently suitable for standard interventions in which no major complications are expected. However, no instruments can be introduced through the infusion trocar. If an instrument trocar is needed at the location where the infusion trocar is being used, the infusion trocar has to be removed and applied again at another location. This also requires new insertion of the instrument trocar.

A further device is known from WO 01/68016. In the latter, an orientation device is inserted into the eye by means of an insertion instrument. The orientation device has a cylindrical design and principally comprises a flange with a cam. Further instruments, for example a cutting instrument, can then be inserted into the eye via the orientation device. The insertion instrument is connected to the orientation device via the cam provided on the latter. After the orientation device has been successfully applied, this connection can be undone, and the insertion tool can be removed. The orientation device can then receive the further instruments. A disadvantage is that, when removing the insertion instrument by actuating the lever for release from the cam, transverse forces may arise, such that the opening in the eye is enlarged. This compromises the subsequent healing process, for example.

### DISCLOSURE OF THE INVENTION

Starting out from this prior art, the object of the invention is to create a device that provides improved compatibility between the pilot tube and the instruments that are to be inserted into the pilot tube, for example infusion lines or illuminating devices.

A further aim of the present invention is to configure said device in such a way that it allows a surgeon the greatest possible flexibility in terms of its use, such that surgical interventions on an eye can be performed with great efficiency.

This object is achieved by a device having the features of Patent claim 1. Advantageous embodiments of the invention are set out in the dependent claims.

Accordingly, a pilot tube attachment can be connected by coupling to a pilot tube inserted into an eye and has a centre axis. The pilot tube attachment comprises an instrument guide and a clamp unit. The clamp unit comprises at least two catch elements which are arranged symmetrically with respect to the centre axis and which can be actuated by respectively associated pressure tabs via a transition leading into the instrument guide.

Such a pilot tube attachment can be connected in a particularly efficient manner to an inserted pilot tube or to a pilot tube that is to be inserted. In addition, the device permits simple separation and replacement of the pilot tube attachment.

The pressure tabs and the catch elements are preferably arranged rotationally symmetrically about the centre axis and are coaxial with respect to one another.

By means of a coaxial arrangement, the pilot tube attachment is easy to manipulate, and the occurrence of radial forces during its manipulation can be avoided.

The transition between instrument guide and clamp unit is preferably a disc.

Preferably, the catch elements, on their side inclined towards the main axis, at least partially have a locking notch.

The locking notch is particularly advantageous, since a particularly secure connection between pilot tube and pilot tube attachment is afforded by means of the locking notch.

Preferably, the catch elements, on their side directed away from the main axis, at least partially have a gripping groove.

The gripping groove allows the person using such a pilot tube attachment to securely grip the latter.

The instrument guide preferably has the form of an integrated connector piece. The connector piece can be connected to an instrument from the group of infusion line, illuminating unit, cutting instrument, diathermy instrument, illuminating instrument or optical waveguide.

The instrument guide preferably has the form of a connector insert. The connector insert can be connected to an instrument from the group of infusion line, illuminating unit, cutting instrument, diathermy instrument, illuminating instrument or optical waveguide.

A set preferably comprises at least one pilot tube attachment, preferably at least three pilot tube attachments, in particular for an instrument from the group of infusion line, illuminating unit, cutting instrument, diathermy instrument or optical waveguide, and at least one pilot tube, preferably three identical pilot tubes. The pilot tubes consist of a cannula and a flange which, at its end directed away from the cannula, has a complementary bead for the catch elements.

Between pilot tube and pilot tube attachment, there is preferably at least one seal for producing a fluid-tight connection between pilot tube and pilot tube attachment.

A pilot tube attachment and/or a set is preferably used for applying an infusion or for introducing an optical waveguide into the interior of the eye or for introducing a surgical tool into the interior of the eye.

Further advantageous embodiments are characterized in the dependent claims.

### BRIEF DESCRIPTION OF THE DRAWINGS

The invention is described in more detail below with reference to the drawings, in which:

FIG. 1 shows a cross-sectional view of an eye, together with a device according to a first illustrative embodiment of the present invention;

FIG. 2 shows a detail of FIG. 1;

FIG. 3 shows a perspective view of a pilot tube attachment according to the present invention, from above;

FIG. 4 shows a perspective view of the pilot tube attachment according to FIG. 3, from below;

FIG. 5 shows a cross-sectional view of the pilot tube attachment according to FIGS. 3 and 4;

FIG. 6 shows a cross-sectional view of an eye, together with a device according to a second illustrative embodiment of the present invention;

FIG. 7 shows a detail of FIG. 6;

FIG. 8 shows a perspective view of a pilot tube attachment according to a second illustrative embodiment of the present invention, from above;

FIG. 9 shows a perspective view of the pilot tube attachment according to FIG. 8, from below;

FIG. 10 shows a cross-sectional view of the pilot tube attachment according to FIGS. 8 and 9; and

FIG. 11 shows an insert piece for insertion into the pilot tube attachment.

### DESCRIPTION OF AN ILLUSTRATIVE EMBODIMENT

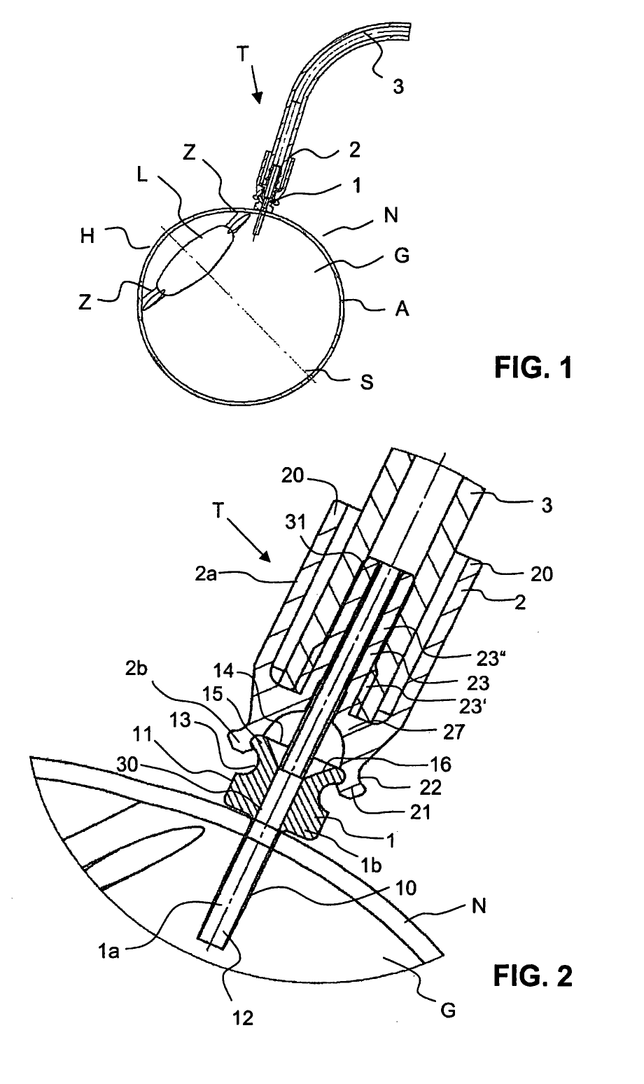

FIG. 1 shows a cross-sectional view of a human eye, together with a device T according to a first illustrative embodiment inserted into the eye. The individual elements of the eye are represented schematically. These include the vitreous body G, the cornea H, the lens L, the retina N, the optic nerve S and the zonular fibres Z. In the present illustrative embodiment, the device T is inserted in the area adjacent to the area of the zonular fibres Z. However, the device T can also be inserted into the eye at other locations. The device T according to the invention principally comprises a pilot tube 1, a pilot tube attachment 2 and, for example, an infusion line 3. The pilot tube 1 can, for example, also be designated as a trocar, in which case the pilot tube attachment 2 is then designated as a trocar clip. The infusion line 3 is mentioned here only by way of example and can also be, for example, an optical waveguide or, indeed, any desired instrument.

FIG. 2 shows a detailed sectional view of the device T according to the invention.

The pilot tube 1 is in principle rotationally symmetrical about a centre axis 17 and comprises a first section 1a and a second section 1b. In the first section 1a, the pilot tube 1 has a cylindrical outer shape. This first section 1a can also be designated as a cannula 10. The second section 1b is composed of a flange 11 formed integrally on the cannula 10. The flange 11 has a greater external diameter than the cannula 10. The surface of the flange 11 directed towards the eye is flat.

An opening 12 extends through the pilot tube 1 along the longitudinal axis or centre axis 17 and thus forms a channel through the first section 1a and the second section 1b. In the area where the opening issues from the flange 11, the opening 12 widens via a conical outlet 16. The conical shape is advantageous, since this permits simple and safe insertion of instruments into the opening 12 of the pilot tube 1.

On its outwardly directed surface, the flange 11 of the second section 1b has a groove 13 extending at least partially or completely about its circumference. The arrangement of the groove 13 at a distance from the surface 14 of the flange 11 means that a kind of circumferential bead 15 is formed. The circumferential bead 15 can be used as a locking means for connecting the pilot tube 1 to the pilot tube attachment 2.

By virtue of the described configuration of the circumferential groove 13, which can also be designated as a forceps groove, the surgeon and the persons assisting the surgeon are able to securely grip the pilot tube 1 with forceps. The pilot tube 1 can be mounted on a mandrel (not shown) and, by means of a force applied via the mandrel, can be inserted into the eye through an opening in the surface of the eye. The opening is preferably an incision made at an angle of 20° to 70°, measured from the line perpendicular to the surface of the eye. An angle of between 30° and 60° is particularly preferred. The incision is made in the surface of the eye preferably by means of a surgical cutting instrument, such as a scalpel. The angled direction of the incision has the effect that the opening in the surface of the eye remains as small as possible, or a kind of flap is formed which then effectively closes the opening after removal of the pilot tube 1. This results in what is, for the patient, an advantageous wound closure. This subsequently allows an accelerated healing of the opening after the intervention. The pilot tube 1 is preferably inserted in the direction of the incision or in a tangential direction.

The mandrel, however, can also be part of an insertion tool (not shown) for inserting the pilot tube 1. In addition, however, the pilot tube can also be inserted by hand or using forceps.

It is also possible for a cutting instrument, for example a blade, to first be inserted into the opening 12 of the pilot tube 1. The required opening can then be cut with this cutting instrument, such that the pilot tube can then be inserted into the eye. This therefore means that the opening can be formed in the tissue in a single step and that the small pilot tube 1 can be inserted at the same time.

As can be seen from FIG. 2, the pilot tube 1 is inserted with the cannula 10 into the eye. The pilot tube 1 can be inserted into the eye until the flange 11 lies on the surface of the eye. The first section 1a has a length that allows the cannula to penetrate into the vitreous body G of the eye.

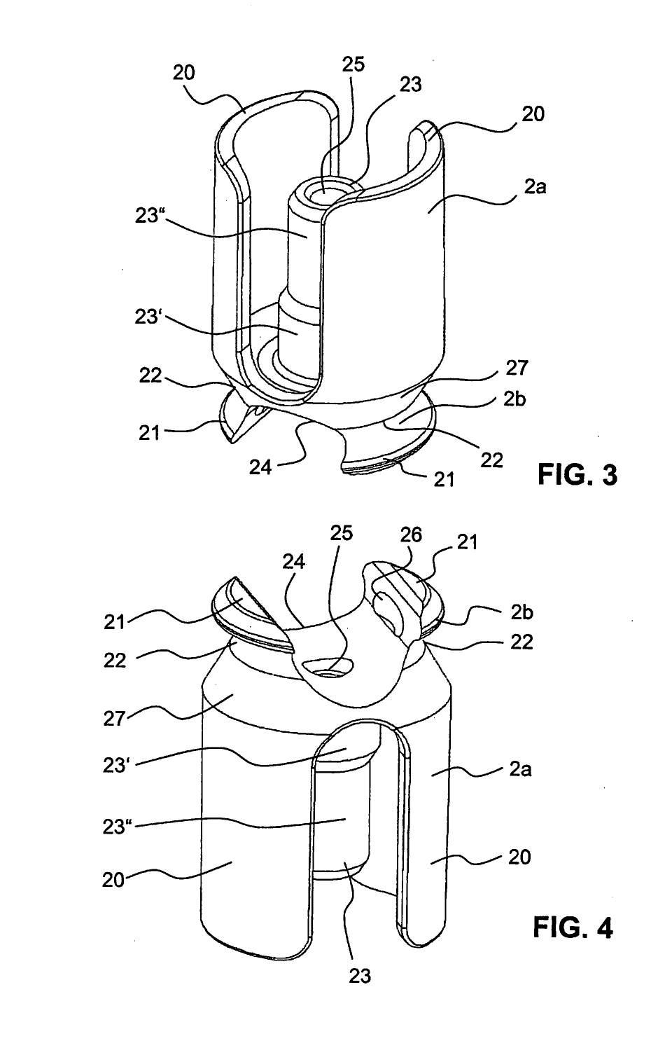

The pilot tube attachment 2 according to a first illustrative embodiment is shown in FIGS. 2 to 5 in various views. The pilot tube attachment 2 comprises an instrument guide area or first upper section 2a, and a clamp area or a second lower section 2b, the two sections being separated by a circular cylindrical disc 27. The pilot tube attachment 2 preferably has a rotationally symmetrical design.

The upper section 2a comprises several, in this case two, outer pressure tabs 20, and an inner instrument connector piece or instrument guide 23 designed as a plug. The plug 23 can also be designated as an integrated connector piece 23. Both the plug 23 and the pressure tabs 20 are formed integrally on the top face of the circular cylindrical disc 27 and extend perpendicular to this top face.

The plug 23 is concentric with respect to the circular cylindrical disc 27 and has a hollow cylinder design. That is to say, the plug has an opening 25. The centre axis 17, which is also the centre axis of the pilot tube attachment 2, extends through the centre of the circular cylindrical disc 27 and through the plug 23. In a first area 23′ located nearer to the circular cylindrical disc 27, the plug 23 has a greater external diameter than it does in the upper area 23″. The transition from the upper area 23″ to the lower area 23′ has a conical shape.

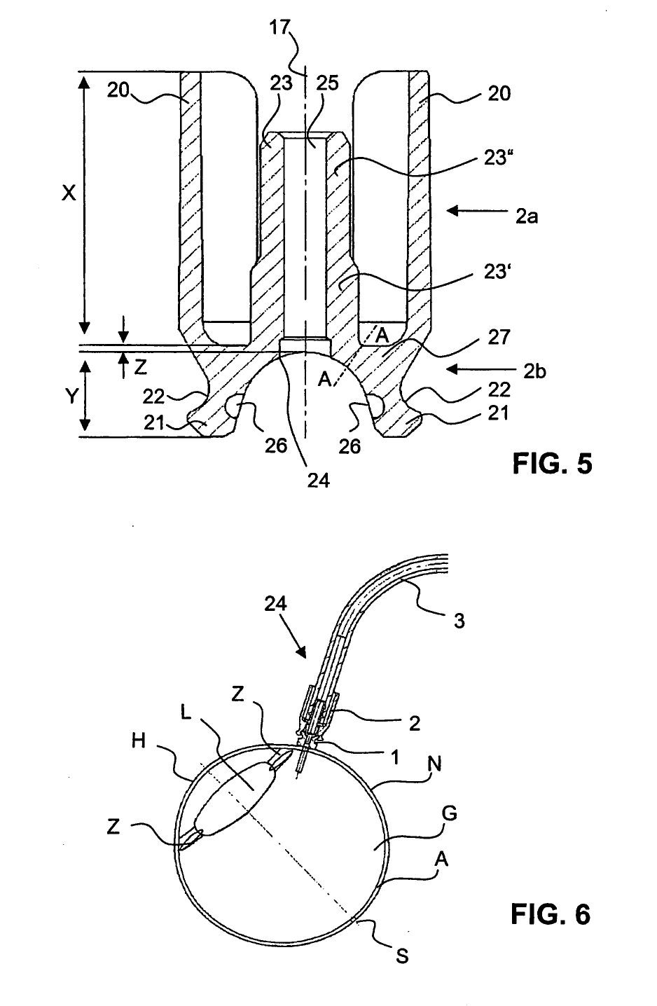

The pressure tabs 20 are formed integrally on the outer edge of the disc 27, preferably rotationally symmetrically, and in the present illustrative embodiment they extend about an arc length that delimits an angle of 90° to 150°. Smaller angles are also possible. That is to say, therefore, that in the illustrative embodiment shown here the instrument connector piece 23 is arranged between the two pressure tabs 20. As can be seen in FIG. 5, the pressure tabs 20 have a greater height than the plug 23.

The lower section 2b in principle comprises several, in this case two, catch elements 21. The catch elements 21 are formed integrally on the lower face of the circular cylindrical disc 27. The catch elements 21 are arranged in such a way that they are arranged underneath and in the same direction as the pressure tabs 20. The number of catch elements 21 is therefore preferably equal to the number of pressure tabs 20. An intermediate area 24 between the catch elements 21 is configured as a recess. The recess 24 is designed, in particular in terms of its dimensions, in such a way that a small cross-sectional surface is obtained at the location indicated, in FIG. 5, by a line A-A. The expression “small cross-sectional surface” is to be understood as meaning that the resulting cross section has the least possible flexural strength and that, therefore, a deformation is possible in this area, even under slight forces. The deformation is to be understood here as elastic deformation. To achieve this result, however, the circular cylindrical disc 27 also has to have the smallest possible thickness. Because of the small thickness of the cross-section A-A of the disc 27 relevant for the movement, the connection between disc 27 and catch elements 21 and pressure tabs 20 can also be designated as a film hinge. By virtue of the design of the pressure tab 20 and the catch element 21, the relative deformation between pressure tab 20 and catch element 21, upon actuation of the pilot tube attachment 2, remains relatively small or is non-existent. In other words, this means that the pressure tab 20 and the catch element 21 remain rigid with respect to one another during actuation. Accordingly, it is preferably only the cross section A-A that deforms.

In its lower area, that is to say in the area in which the opening 25 issues into the clearance space 24, said opening 25 has a slightly greater diameter than in the area of the instrument connector piece 23. This greater diameter can also be designated as a widening. This widening serves, for example, as an insertion aid for pressing a cannula into place. Moreover, this widening can also be used as an adhesion gap, if a cannula is to be adhesively bonded to the pilot tube attachment.

On the side directed towards the intermediate area 24, the catch elements 21 also have locking notches 26. The locking notches preferably have a surface complementing the circumferential bead 15 of the pilot tube 1. On the outer side, the catch elements 21 also have recesses or forceps grooves 22. These recesses or forceps grooves 22 allow the pilot tube attachment 2 to be gripped by forceps. Moreover, by virtue of an effect described below, the recesses 22 have proven advantageous for the deformation of the catch elements 21.

A force applied to the two pressure tabs 20 in such a way that the two pressure tabs 20 are moved towards one another has the effect that the catch elements 21 lying opposite the pressure tabs 20 are spread open. The pressure tabs 20 can be pressed together manually with two fingers or also by means of a forceps or other tool. When the force is not applied, that is to say in the state free of any force, the two pressure tabs 20, and accordingly also the catch elements 21, return to their original position.

In the axial direction, the pressure tabs 20 have a greater length than the catch elements 21. The length of the pressure tabs 20 is designated here by X, and the length of the catch elements 21 is designated by Y. A dimension Z lying between these represents the approximate thickness of the disc 27. The ratio X:Y is preferably between 10:1 and 1:1, particularly preferably between 6:1 and 2:1. The length ratios described here are particularly advantageous since, because of a lever action via the disc 27, the pressure tabs 20 have to be pressed together over a suitably large distance such that the catch elements move outward by a suitably smaller distance. This means that inadvertent release of the pilot tube attachment 2 from the pilot tube 1 is avoided.

In the state when spread open, the pilot tube attachment 2 can then be pushed with the lower section 2b over the pilot tube 1. In doing so, the locking notches 26 of the pilot tube attachment 2 come to rest on the circumferential bead 15 of the pilot tube 1. This means that, in the state when spread open, the internal diameter of the pilot tube attachment 2 in the lower area 2b is greater than the external diameter of the pilot tube 1. This therefore prevents an axial force from acting on the pilot tube 1 when the pilot tube attachment 2 is connected to the pilot tube 1. Also, by virtue of the symmetrical introduction of force, no radial forces are applied to the pilot tube 1. As soon as the user stops applying the force to the pressure tabs 20, the catch elements 21 return, as has been described above, to their original position. The catch elements 21 then touch the circumferential bead 15, such that a form-fit connection is established. The circumferential bead is then concentrically surrounded by the catch elements 21. When the pilot tube 1 and the pilot tube attachment 2 are in engagement with one another in this way, this is also referred to as the assembled state.

Depending on the dimensions of the circumferential bead 15 and of the catch elements, the form-fit connection is supported by a force-fit connection.

The external diameter of the circumferential bead 15 can, for example, be chosen greater than the internal diameter of the locking notches 21 in the state when free of force. The result of this is that, in the assembled state, a clamping force is exerted, owing to the mechanical configuration, from the pilot tube attachment 2 onto the pilot tube 1. There is therefore a force fit and a form fit. If the resulting frictional force, which results from the clamping force and material pairing or surface nature of the pilot tube 1 and of the pilot tube attachment, is greater than the static frictional force to be overcome in the radial direction, a rotation movement of the pilot tube attachment 2 about the pilot tube 1 is avoided. Because of the dimensioning of the diameter of the circumferential bead 15 and of the internal diameter of the catch elements, the connection between the pilot tube 1 and the pilot tube attachment 2 is both a force-fit and also a form-fit connection.

If the diameter of the circumferential bead 15 is chosen smaller than the internal diameter of the locking notches 21 in the state when free of force, no residual force results. In this case, therefore, a rotation movement of the pilot tube attachment 2 about the pilot tube 1 is permitted. This therefore involves a form-fit connection.

By virtue of the rotationally symmetrical design, the pilot tube attachment 2 with respect to the pilot tube 1 can assume any desired position in relation to the rotation angle between pilot tube attachment 2 and pilot tube 1. This is particularly advantageous, since it ensures the surgeon a particularly efficient and flexible placement of the pilot tube attachment 2. The same advantage can be achieved with a rotationally symmetrical pilot tube and a rectangular pilot tube attachment if, on two opposite sides of the rectangle, two pressure tabs 20 are connected to catch elements 21 via in this case a rectangular disc 27.

By renewed application of a force to the pressure tabs 20, the catch elements 21 are once again spread open. In this way, the pilot tube attachment 2 can be easily removed from the pilot tube 1. In this case too, no axial and radial forces are applied to the pilot tube 1. If the entire device T is to be removed from the eye, this can also be done without removing the pilot tube attachment 2 from the pilot tube 1. A tensile force acting on the pilot tube attachment 2 can be transmitted directly to the pilot tube 1 by way of the connection between pilot tube attachment 2 and pilot tube 1. The pilot tube 1 is then removed from the opening in the eye by the tensile force. The tensile force can either be effected directly by hand on the pressure tabs 20 or alternatively via forceps engaged in the forceps grooves 22. The removal procedure, in the case of infusion, thus takes place without loss of liquid, because the pilot tube 1 and the pilot tube attachment 2 here form one unit.

The process of connection and separation of the pilot tube attachment 2 with a pilot tube 1 is, as has been described above, particularly advantageous, since no axial forces (tensile forces or pressure forces) arise in the two processes. The surgeon can thus connect a pilot tube attachment 2 to a pilot tube 1 in a simple manner and then separate them again. This permits flexible use of a pilot tube attachment 2 on different pilot tubes 1 inserted in the eye.

As has already been mentioned, the instrument connector piece has an opening 25 that extends through the plug 23 as far as the intermediate area 24. Articles or fluids can be guided through this opening from the first section 2a into the second section 2b.

As is shown in FIG. 2, an infusion line 3, for example, can be fitted over the plug 23. The line additionally comprises a cannula 31. The cannula 31 has a length which is dimensioned such that the cannula 31 can protrude into the opening 12 of the pilot tube 1. PVC or silicone hoses are suitable in particular as infusion lines. A pilot tube attachment 2 for an infusion is designated as an infusion pilot tube attachment.

For example, the radial play between the external diameter of the cannula 31 and the internal diameter of the pilot tube 1 can be a maximum of 0.02 mm. This results in a sufficient leaktightness for this application. Liquid can then only emerge in the form of droplets.

Alternatively, the pilot tube attachment 2 can also be connected to an optical waveguide for illuminating the interior of the eye. A pilot tube attachment 2 for illumination is designated as an illumination pilot tube attachment. An optical waveguide then protrudes for example into the first area 1a of the pilot tube 1.

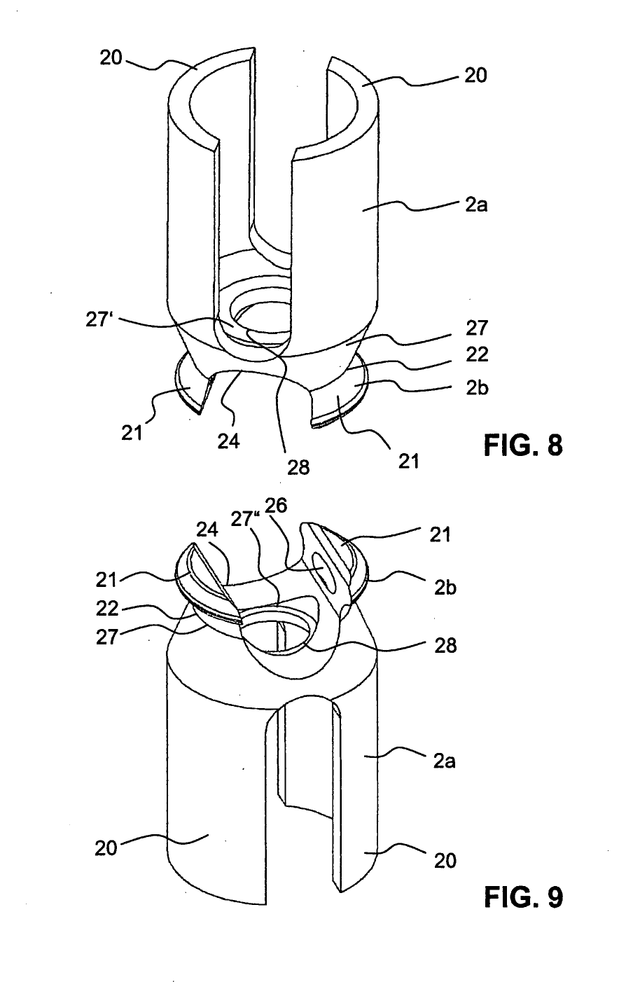

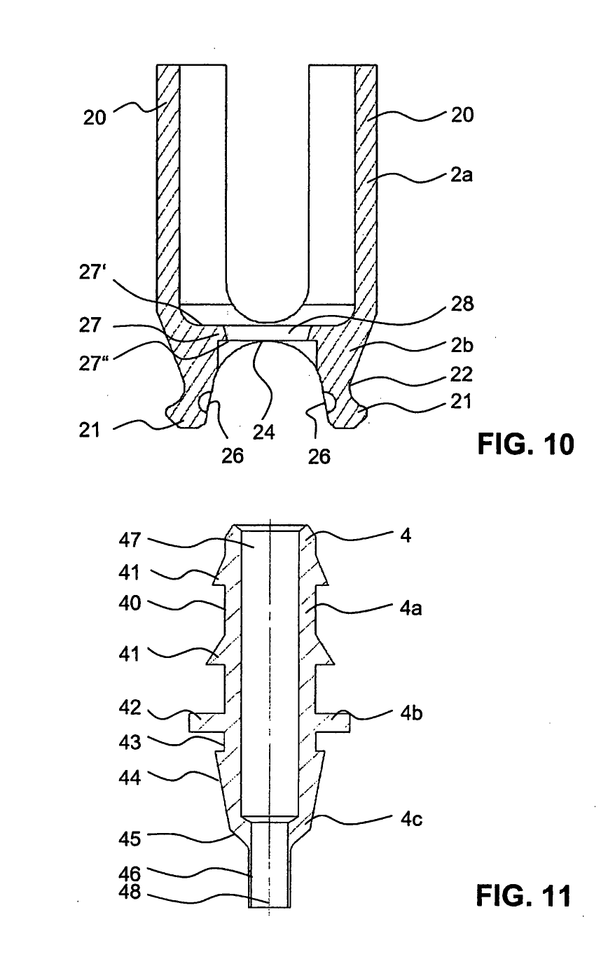

FIGS. 6 to 11 show depictions of the device according to the invention with the pilot tube 1 and the pilot tube attachment 2 according to a second illustrative embodiment of the present invention. Here, the pilot tube 1 is identical in design to the pilot tube 1 of the first illustrative embodiment. In addition, the device in this illustrative embodiment comprises a connector piece 4. The same parts are provided with identical reference signs.

FIG. 11 shows the connector insert 4. The connector insert 4 has a substantially cylindrical design and has, in the axial direction, a first section 4a, a second section 4b and a third section 4c.

The first section 4a can be connected, for example, to an infusion line or an optical waveguide. On the substantially cylindrical outer face 40, the first section 4a has a plurality of elevations 41, in this case two elevations 41. The outer face 40 can also be designated as an instrument guide. These elevations 41 extend in a complete ring shape or are interrupted and, in cross section, have the shape of a wedge and, by virtue of their shape, they increase the retaining force between infusion line 3 and connector insert 4.

The second section 4b comprises a flange 42. The flange 42 has a diameter that is greater than the diameter of the first section. By way of a cylindrical outer shape 43, with a smaller diameter than the diameter of the flange 42, the second section 4b merges into the third section 4c.

The third section 4c forms the endpiece of the connector insert 4. The third section 4c in principle comprises two cones 44, 45 and a cylindrical endpiece 46. The first cone 44 extends from the cylindrical outer shape 43. This cone 44 has, at the start, a greater diameter than the cylindrical outer shape 43. The diameter of the cone 44 decreases in a constant manner. The cone 45 is formed integrally on the cone 44 and also tapers. However, the cone 45 tapers more steeply than the cone 44. A cylindrical plug 46 forms the endpiece of the third section.

Moreover, the connector insert 4 has an opening 47 that extends along a centre axis 48 through all three sections 4a, 4b, 4c.

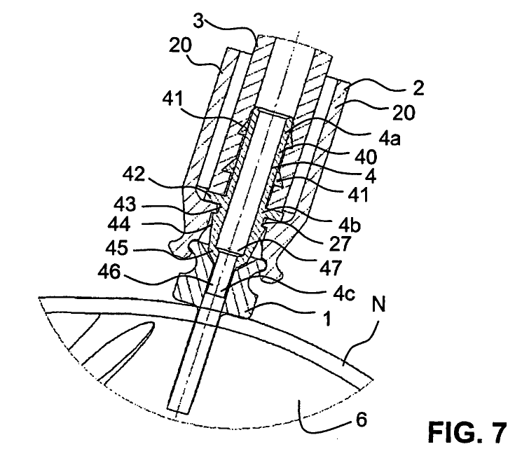

FIGS. 7 to 10 show the pilot tube attachment 2 according to the second illustrative embodiment. In analogy with the first illustrative embodiment, the pilot tube attachment 2 likewise has an upper section 2a and a lower section 2b. The upper section 2a and the lower section 2b can also be designated as a clamp unit. As in the first illustrative embodiment, the upper section 2a has several pressure tabs 20, in this case two pressure tabs 20. An opening 28 passes through the circular cylindrical disc 27. The opening can also be designated as an instrument guide. The opening 28 has in this case a shape that narrows from the upper section 2a to the lower section 2b. The connector insert 4 can be introduced into this opening 28. The narrowing shape and the conical configuration of the connector insert 4 in the third section 4c supports the process of insertion. One face of the flange 42 comes to lie on the upper face 27′ of the circular cylindrical disc 27, and the cylindrical outer shape 43 comes to rest on the surface of the opening 28. The transition from the cylindrical outer shape 43 to the cone 44 rests on the lower face 27″ of the circular cylindrical disc 27 in the inserted state. The connector insert 4 is thus secured against axial movements, but it can still be separated from the pilot tube attachment 2 in the event of substantial axial forces, for example as occur upon desired separation of connector insert 4 and pilot tube attachment 2. The above-described effect of the film hinge is still present here. The circular cylindrical disc 27 can in this case act as the film hinge. The circular cylindrical disc can also be described as a torus or as similar to a torus.

FIG. 7 shows the pilot tube attachment 2 with the connector insert 4 attached to the pilot tube 1. It will also be seen here that the connector insert according to the second illustrative embodiment is designed in such a way that the cone 45 comes to rest on the conical outlet 16 of the pilot tube 1. The cylindrical plug 46 protrudes into the opening 12 of the pilot tube 1. By means of the cone 45 resting on the outlet 16, and the hollow cylindrical plug 46 protruding into the opening 12, a liquid-tight connection is provided between connector insert 4 and pilot tube 1.

In an operation on an eye, the surgeon typically uses several, in particular three, pilot tubes 1 according to the present invention. The pilot tubes 1 perform a wide variety of functions. The nature of the connection between pilot tube 1 and pilot tube attachment 2 allows the surgeon, or the surgeon's assistant, to easily and efficiently change the pilot tube attachment 2. If, instead of the pilot tube attachment with the infusion line, a pilot tube attachment 2 is to be used with the illuminating device, the pilot tube attachment with the infusion line can easily be replaced by the pilot tube attachment with the illuminating device.

The pilot tube attachments 2 according to the present invention are preferably made of plastic, in particular polycarbonate or polyethylene. The pilot tube attachment 2 is preferably produced by an injection-moulding procedure. The plastic used is particularly preferably transparent and thus allows the surgeon a better view of the incision site.

In further embodiments, the pilot tube attachment 2 can also be made of another material, for example metal. In particular, use may be made for example of stainless steel, titanium or titanium alloys. Other biocompatible metals and materials are also conceivable.

The pilot tubes are preferably made of a metal, for example stainless steel, titanium or titanium alloys. Other biocompatible metals and materials are also conceivable. Various plastics, for example polycarbonate or polyethylene, can also be used.

In other illustrative embodiments not shown here, other instruments, for example cutting tools, or other liquid transfer devices can be secured on the pilot tube 1. The cutting tool can be connected to the pilot tube attachment 2 or can be integrated in the latter.

In another illustrative embodiment not shown here, the pilot tube attachment 2 is provided, in its clearance space 24, with a plug that is integrally formed on the lower face of the cylindrical disc. With a pilot tube attachment of this kind, an opening 12 in a pilot tube can be closed in a liquid-tight manner. To increase the sealing action, the plug can be designed in such a way that parts of the plug come to rest on the conical outlet 16. It is additionally possible to provide elastic elements, such as O-rings, which additionally increase the sealing action.

### LIST OF REFERENCE SIGNS

| A | sclera |
|---|---|
| G | vitreous body |
| H | cornea |
| L | lens |
| N | retina |
| S | optic nerve |
| Z | zonular fibres |
| 1 | pilot tube |
| 2 | pilot tube attachment |
| 3 | infusion line |
| 4 | connector insert |
| 10 | cannula |
| 11 | flange |
| 12 | opening |
| 13 | circumferential groove |
| 14 | top face of flange |
| 15 | circumferential bead |
| 16 | conical outlet |
| 17 | centre axis |
| 20 | pressure tabs |
| 21 | catch elements |
| 22 | forceps groove/recess |
| 23 | instrument connector piece |
| 24 | clearance space |
| 25 | opening |
| 26 | locking notches |
| 27 | circular cylindrical disc |
| 28 | opening |
| 30 | seal |
| 31 | cannula |
| 40 | outer face |
| 41 | elevations |
| 42 | flange |
| 43 | cylindrical outer shape |
| 44 | cone |
| 45 | cone |
| 46 | plug |
| 47 | opening |
| 48 | centre axis |

1-10. (canceled)

11: A pilot tube attachment adapted to be connected by coupling to a pilot tube inserted into an eye and having a centre axis, the attachment comprising:
a central instrument guide; and
a clamp unit having at least two catch elements which are arranged symmetrically with respect to the centre axis, the catch elements having associated pressure tabs to actuate the clamp unit via a transition leading into the instrument guide.

12: The pilot tube attachment according to claim 11, wherein the pressure tabs and the catch elements are arranged rotationally symmetrically about the centre axis and are coaxial with respect to one another.

13: The pilot tube attachment according to claim 11, wherein the transition between instrument guide and clamp unit is a disc.

14: The pilot tube attachment according to claim 11, wherein the catch elements, on their side inclined towards the centre axis, at least partially have a locking notch.

15: The pilot tube attachment according to claim 11, wherein the catch elements, on their side directed away from the centre axis, at least partially have a gripping groove.

16: The pilot tube attachment according to claim 11, wherein the instrument guide has the form of an integrated connector piece, wherein the connector piece is connected to an instrument via an infusion line, illuminating unit, cutting instrument, diathermy instrument, illuminating instrument or optical waveguide.

17: The pilot tube attachment according to claim 1, wherein the instrument guide has the form of a connector insert, wherein the insert is adapted to be connected to an instrument via an infusion line, illuminating unit, cutting instrument, diathermy instrument, illuminating instrument or optical waveguide.

18: A set with at least one pair of a one pilot tube and a pilot tube attachment adapted to be connected by coupling to said pilot tube to be inserted into an eye and having a centre axis, the pilot tube attachment comprising:
a central instrument guide; and
a clamp unit having at least two catch elements which are arranged symmetrically with respect to the centre axis, the catch elements having associated pressure tabs to actuate the clamp unit via a transition leading into the instrument guide,
the pilot tube comprising:
a cannula; and
a flange having a complementary bead at its end directed away from the cannula for the catch elements.

19: The set according to claim 18, wherein there is at least one seal for producing a fluid-tight connection between the pilot tube and the pilot tube attachment.

20: The set according to claim 18, wherein at least three pairs of the pilot tube and pilot tube attachment are provided.

21: The set according to claim 18, wherein the set is for use with an infusion line, illuminating unit, cutting instrument, diathermy instrument or optical waveguide.
